(19) United States
(12) Patent Application Publication
(10) Pub. No.: US 2002/0072601 A1
(43) Pub. Date: Jun. 13, 2002

(54) Method for forming amino acid derivatives from tricyclic diketopiperazines

(75) Inventors: Lon J. Mathias, Purvis, MS (US); Dennis A. Parrish, Hattiesburg, MS (US); Louis Somlai, Hattiesburg, MS (US)

Correspondence Address: HOWREY SIMON ARNOLD & WHITE, 750 BERING DRIVE, HOUSTON, TX, 77057-2198, US

(73) Assignee: University of Southern Mississippi

(21) Appl. No.: 09/932,849

(22) Filed: Aug. 17, 2001

Related U.S. Application Data
(60) Provisional application No. 60/226,144, filed on Aug. 18, 2000.

(51) Int. Cl. C07D 413/02; C 07D 4 3/02; C07D 265/32; C07D 205/08; C 07D 2 7/26

(52) U.S. Cl. 540/357; 544/78; 544/171; 548/519; 548/543

(57) ABSTRACT

Amino acid derivatives are formed by reacting a tricyclic diketopiperazine with a nucleophillic compound. In a preferred embodiment, pyroglutamic diketopiperazine is reacted with an amine or an alcohol which opens the six member ring.

This application claims the benefit of U.S. Provisional Application S.No. 60/226,144, filed Aug. 18, 2000.

### BACKGROUND OF THE INVENTION

2,5-Diketopiperazines (DKP), cyclic dimers of amino acids, occur frequently in nature as degradation products of peptides and proteins. The ease of formation of DKPs has lead peptide chemists to develop solid phase techniques to both avoid their formation and to synthesize them for further study. DKPs have been studied for their biological activity, self-organization, gelation of organic solvents, and potential for drug delivery.

DKPs contain two cis-amide linkages in an anti-parallel alignment (Structure 1).

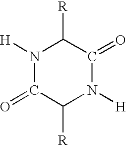

### Structure 1. Structure of Symmetrical Diketopiperazine

The two cis-amide functional groups inherent in DKP rings are expected to impart significant improvements in material properties, as well as provide the potential for property modification of composites and biological systems.

Pyroglutamic acid, the internally protected form of glutamic acid, is commonly found in biological systems. There has been recent interest in peptide based drugs incorporating constrained amino acid residues for the purpose of strategic receptor interaction. In addition, derivatives of pyroglutamic acid have been studied as antiamnesic agents and as treatments for asthma and rheumatoid arthritis. In organic synthesis, pyroglutamic acid is frequently used as a starting material in the preparation of enantiomerically pure compounds, such as chiral auxiliaries in aymmetric synthesis, and as a precursor for other amino acids.

Self-assembling supramolecular polymers based upon non-covalent bonding has attracted considerable interest due to the potential applications in the areas of molecular devices and biological mimics. One recent report in the literature indicates that pyroglutamic acid may be useful in forming molecular associations. See, Tsiourvas, Dimitris; Paleos, Constantinos M.; Skoulios, Antoine, “Smectic Liquid Crystalline Character of N-Alkylammonium Pyroglutamates,” Liquid Crystals, 1999, 26, pp. 953-957. In that paper, the authors report that N-alkylammonium salts of pyroglutamic acid display smectic liquid crystalline character. In comparison, the authors note that while ammonium salts of poly(acrylic acid) and poly(maleic acid) show liquid crystalline character, the monomeric analogues do not. Since the N-alkylammonium pyroglutamates are not polymeric, the observation of liquid crystalline behavior is believed to result from the combination of van der Waals association of the alkyl substituents and hydrogen bonding interactions of the pyroglutamate head groups.

We have been pursuing the synthesis of polymers which contain DKP units in the polymer backbone. In an effort to accomplish this task efficiently, pyroglutamic diketopiperazine (PyDKP) was investigated as a monomer for ring-opening polymerization reactions at the five-membered rings (Reaction 1). During the investigation,

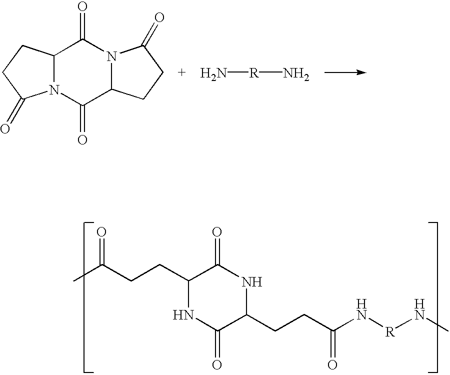

### Reaction 1. Proposed Ring-opening Polymerization

Japanese patent literature discussing the use of PyDKP in this exact manner was uncovered. See, JP 43026198, 1968. However, we found that nucleophilic compounds, both mono-functional and multi-functional, react with PyDKP at the six-membered ring to give derivatives of pyroglutamic acid, not DKP as previously reported (Reaction 2).

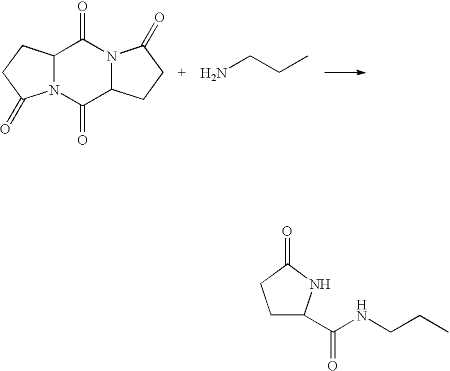

### Reaction 2. Six-membered Ring-opening Reaction of PyDKP

### SUMMARY OF THE INVENTION

The present invention provides methods for the preparation of peptides, organic solvent gelators, and supramolecular polymers derived from the ring-opening reaction of PyDKP. In a preferred embodiment, the present invention comprises a process for forming amino acid derivatives from tricyclic diketopiperazines. The diketopiperazines are reacted with nucleophillic compounds to open the 6 member ring. In preferred embodiments, pyroglutamic diketopiperazine is reacted with various amides or alcohols.

### BRIEF DESCRIPTION OF THE DRAWINGS

FIG. 1 is a graph of differential scanning calorimetry thermograms of the compounds of Example 2.

FIG. 2 is a graph of the thermogravimetric analysis of the compounds of Example 2.

### DETAILED DESCRIPTION OF THE PREFERRED EMBODIMENTS

There are several different DKP compounds that are useful in practicing the present invention. The compound of particular interest is pyroglutamic diketopiperazine (PyDKP, R=CH₂) (Structure 2).

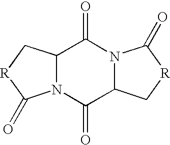

### Structure 2. General Structure of PyDKP

PyDKP may be reacted with various nucleophilic compounds to open the ring structures to prepare compounds that have unique properties. For example, pyroglutamic acid derivatives may be formed by opening the six-membered as shown in Reaction 3.

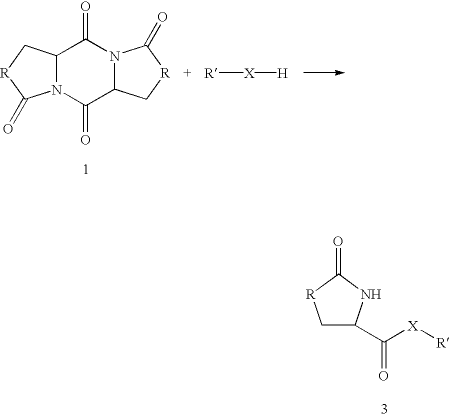

R=nothing, CH₂,

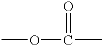

R′=alkyl, alkyl with heteroatoms and multiple functionality, aryl, aryl with heteroatoms and multiple functionality and combinations of these

X=NH, O, S

### Reaction 3. Synthesis of Pyroglutamic Acid Derivatives from DKP

The five-membered rings of PyDKP may also be opened to form 2,5-diketopiperazines with substituents in the 3 and 6 position as shown in Reaction 4.

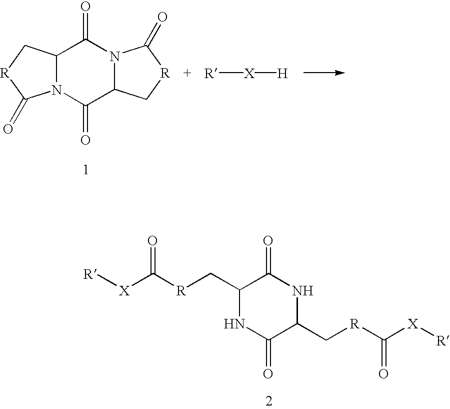

R=nothing, CH₂,

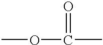

R′=alkyl, alkyl with heteroatoms and multiple functionality, aryl, aryl with heteroatoms and multiple functionality and combinations of these

X, Y=NH and S, and combinations of these

### Reaction 4. Synthesis of Substituted DKPs from DKP

Reactive difunctional nucleophiles may be reacted with PyDKP in a similar procedure to open the six-membered ring to form a class of compounds which form supramolecular polymers through non-covalent bonding (Reaction 5).

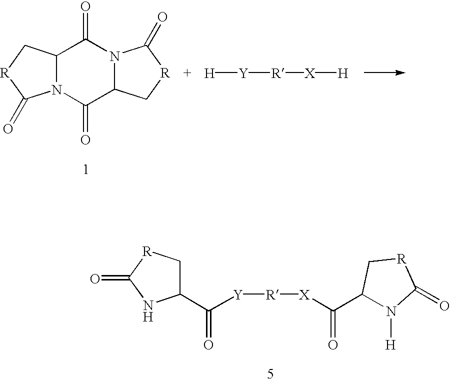

R=nothing, CH₂,

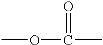

R′=alkyl, alkyl with heteroatoms and multiple functionality, aryl, aryl with heteroatoms and multiple functionality and combinations of these

X, Y=NH, O, S, and combinations of these

### Reaction 5. Formation of Bis-pyroglutamates

Similarly, difunctional compounds that contain nucleophiles of differing reactivity may be used to ring-open PyDKP. The resulting pyroglutamates contain a reactive group that may be used for further modification (Reaction 6). The selective ring-opening of PyDKP provides a one-step route to this class of compounds without the use of protecting groups. Conventional peptide coupling techniques would require three steps or more.

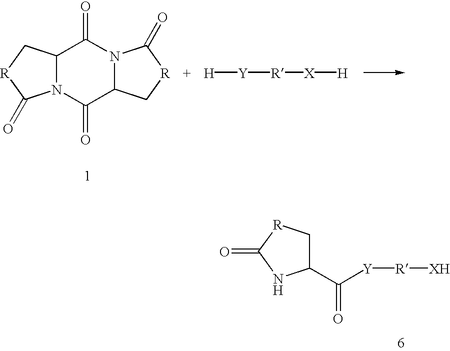

R=nothing, CH₂,

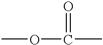

R′=alkyl, alkyl with heteroatoms and multiple functionality, aryl, aryl with heteroatoms and multiple functionality and combinations of these

X, Y=NH, O, S, and combinations of these

### Reaction 6. Formation of Reactive Pyroglutamte Derivatives for Further Modification

The ring-opening reaction of PyDKP with certain nucleophiles (Reaction 3) forms a class of pyroglutamate derivatives that gel organic solvents. The results of gelation studies with N-tetradecyl pyroglutamide is shown in Table 1. In these experiments, the solubility and minimum concentration needed for solvent gelation were determined. Tetradecyl pyroglutamide was placed in various solvents which were then heated to their boiling point, except silicone and soybean oil, which were heated to 150° C. to prevent decomposition of the gelator. In most cases, regardless of partial insolubility in some instances, the various solvents formed immobile gels upon cooling to room temperature. All of the gels were thermoreversible, meaning the gel would revert back to liquid with the application of heat and then reform with cooling.

TABLE 1
Solubility and gel concentration of N-tetradecyl pyroglutamide.

| Solvent | Solubility | Concentration (g/dm³) |
|---|---|---|
| Acetonitrile | +/− | 15 |
| Benzene | + | 100 |
| Butanol | + | 100 |
| Chloroform | +/− | 160 |
| Dichioromethane | +/− | 50 |
| Dimethylacetamide | +/− | — |
| Ethanol | +/− | 30 |
| Hexanol | + | — |
| Methanol | +/− | 30 |
| 1-Propanol | +/− | 50 |
| Trifluoroethanol | − | — |
| Water | − | — |
| Silicone Oil | + | 11 |
| Soybean Oil | + | 11 |

The invention is further illustrated by the following examples.

Experimental Procedure

Solvents and reagents were purchased from Aldrich Chemical Company and used as received. ¹H and ¹³C NMR spectra were obtained in DMSO-d6 using a Bruker AC-200 spectrometer operating at 200.133 MHz for hydrogen and 50.323 MHz for carbon. IR Spectra were recorded on an ATI-Mattson Galaxy 5020 FT-IR spectrometer. Thermal analysis was performed on a TA Instruments SDT 2960 TGA-DTA at 20° C./min under nitrogen and TA DSC 2920 at 10° C./min under nitrogen.

### EXAMPLE 1

Dipropyl-2,5-diketopiperazine-3,6-dipropanamide (2a) and propyl pyroglutamide (3a) were formed by the following reaction as illustrated by Scheme 1.

[chemical structure]

To a 50 mL round bottom flask were added pyroglutamic diketopiperazine (1.00 g, 0.0045 mol), CHCl₃ (10 mL) and a magnetic stir-bar. Propylamine (0.59 g, 0.0099 mol) was added and the reaction mixture was stirred for four hours. The white solid was collected by filtration and dried in vacuo to give 2a. Yield 0.078 g (5.1%); mp 290° C. (decomp); ¹H NMR (DMSO-d₆ with TMS): δ: 7.97 (s, 1H), 7.64 (s, 1H), 3.82 (t, J=5.15 Hz, 2H), 3.03-2.96 (m, 4H), 2.27-2.10 (m, 4H), 1.46-1.34 (m, 4H), 0.84 (t, J=7.35 Hz, 6H). ¹³C NMR (DMSO-d₆ with TMS): δ: 171.1, 167.5, 53.5, 40.1, 30.9, 30.5, 22.0, 11.0. Anal Calcd for C₁₆H₂₈N₄O₄: C, 56.45%; H, 8.29%; N, 16.46%. Found: C, 56.39%; H, 8.23%; N, 16.46%. CHCl₃ was removed from the filtrate in vacuo to give 3a as a white solid. Yield 1.424 g (92.7%); mp 103-106° C.; ¹H NMR (DMSO-d₆ with TMS): δ: 7.93 (s, 1H), 7.77 (s, 1H), 3.98-3.93 (m, 1H), 3.06-2.99 (m, 2H), 2.30-2.05 (m, 3H), 1.89-1.78 (m, 1H), 1.48-1.36 (m, 2H), 0.84 (t, J=7.35 Hz, 3H). ¹³C NMR (DMSO-d₆ with TMS): δ: 177.4, 172.2, 55.9, 40.6, 29.3, 25.4, 22.5, 11.2. Anal Calcd for C₈H₁₄N₂O₂: C, 56.45%;H, 8.29%; N, 16.46%. Found: C, 56.00%; H, 8.27%; N, 16.27%.

Dibenzyl-2,5-diketopiperazine-3,6-dipropanamide (2b) and benzyl pyroglutamide (3b) were also prepared according to Scheme 1. To a 50 mL round bottom flask were added pyroglutamic diketopiperazine (0.989 g, 0.0045 mol), CHCl₃ (20 mL) and a magnetic stir-bar. Benzylamine (1.00 g, 0.0093 mol) was added and the reaction mixture was stirred for 12 hours. The white precipitate was collected by vacuum filtration, mixed with DMF, and filtered once more. The white solid was dried in vacuo to give 2b. Yield 0.132 g (6.8%); mp 264° C. (decomp); ¹H NMR (DMSO-d₆ with TMS): δ: 8.33 (s, 2H), 8.16 (s, 2H), 7.33-7.23 (m, 10H), 4.26 (d, 4H), 3.87 (m, 2H), 2.32-2.17 (m, 4H), 2.03-1.88 (m, 4H). ¹³C NMR (DMSO-d₆ with TMS): δ: 171.5, 167.7, 139.5, 128.3, 127.2, 126.7, 53.5, 42.1, 30.8, 29.1. Anal Calcd for C₂₄H₂₈N₄O₄: C, 66.04%; H, 6.47%; N, 12.84%. Found: C, 65.92%; H, 6.35%; N, 12.71%. CHCl₃ and DMF were removed from the combined filtrates in vacuo to give 3b as a white solid. Yield 1.671 g (86%) white solid; mp 134-137° C.; ¹H NMR (DMSO-d₆ with TMS): δ: 8.51 (s, 1H), 7.87 (s, 1H), 7.35-7.25 (m, 5H), 4.30 (d, 2H), 4.07-4.038 (m, 1H), 2.34-1.86 (m, 4H). ¹³C NMR (DMSO-d₆ with TMS): δ: 177.6, 172.6, 139.3, 127.3, 127.1, 126.9, 56.1, 42.2, 29.4, 25.5. Anal Calcd for C₁₂H₁₄N₂O₂: C, 66.04%; H, 6.47%; N, 12.84%. Found: C, 65.83%; H, 6.55%; N, 12.72%.

The reaction of pyroglutamic anhydride with mono-amine gives both the six-membered and five-membered ring opened products. Opening of the six-membered ring gives the major product in 93% yield. Opening of the five-membered rings gives the minor product in 5% yield. Formation of the minor product can be decreased or essentially eliminated by lowering the reaction temperature. The yield of the minor product can be increased by increasing the reaction temperature.

### EXAMPLE 2

N,N′-Bispyroglutamyl-1,3-propanediamine (5, PDA GLP) was formed by the following reaction as illustrated by Scheme 2.

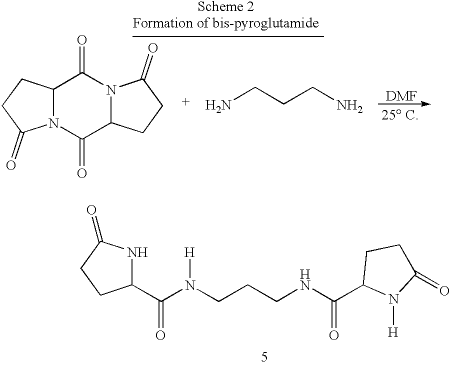

To a 100 mL round bottom flask were added pyroglutamic diketopiperazine (1.00 g, 0.0045 mol), CHCl₃ (40 mL) (DMF may also be used) and a magnetic stir-bar. 1,3-diaminopropane (0.335 g, 0.0045 mol) was added and the reaction mixture was stirred for six hours. The solvent was removed in vacuo and the resulting solid was then dissolved in methanol. The insoluble material was removed by filtration. The filtrate solvent was evaporated in vacuo to give 5. Yield of 5 1.1048 g (82.4%) white solid. ¹H NMR (DMSO-d₆ with TMS): δ: 8.23 (s, 2H), 7.85 (s, 2H), 4.02-3.97 (m, 2H), 3.07-3.03 (m, 4H), 2.29-2.00 (m, 6H), 1.90-1.80 (m, 2H), 1.60-1.50 (m, 2H). ¹³C NMR (DMSO-d₆ with TMS): δ: 177.37, 172.29, 55.80, 36.24, 29.33, 28.84, 25.32.

Similarly prepared were:

N,N′-Bispyroglutamyl-1,3-ethanediamine (EDA GLP). ¹H NMR (DMSO-d₆ with TMS): δ: 8.23 (s, 2H), 7.82 (s, 2H), 4.01-3.97 (m, 2H), 3.15-3.13 (m, 4H), 2.29-2.05 (m 6H), 1.96-1.84 (m, 2H). ¹³C NMR (DMSO-d₆ with TMS): δ: 177.3, 172.5, 55.9, 38.3, 29.3, 25.1.

N,N′-Bispyroglutamyl-2-methyl-1,5-pentanediamine (MPDA GLP). ¹H NMR (DMSO-d₆ with TMS): δ: 8.23 (m, 2H), 7.89 (s, 2H), 4.10-4.02 (m, 2H), 3.04-2.92 (m, 4H), 2.30-2.05 (m, 6H), 1.91-1.82 (m, 2H), 1.58 (m, 1H), 1.43-1.29 (m, 4H). ¹³C NMR (DMSO-d₆ with TMS): δ: 177.4, 172.4, 172.2, 55.8, 44.4, 39.0, 32.5, 31.1, 29.4, 25.4, 17.6.

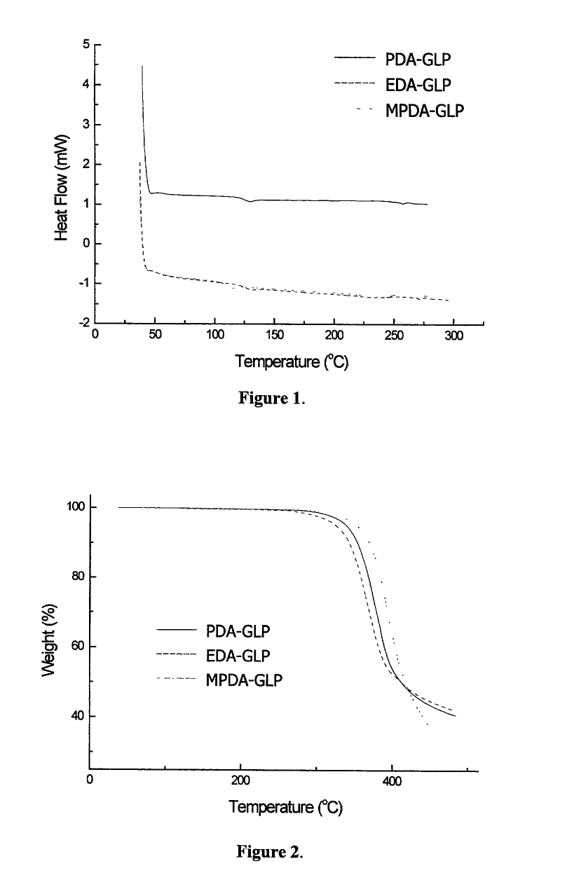

Differential scanning calorimetry (FIG. 1) showed that the bis-pyroglutamates from Scheme 2 had glass transitions. PDA-GLP and EDA-GLP had T_{g}s of 126° C. and 125° C. MPDA-GLP, had the lowest T_{g} at 110° C.

Thermogravimetric analysis (FIG. 2) showed that the compounds were stable up to 300° C. before decomposition began.

Considering the observations of DSC transitions resembling glass transition temperatures, precipitation of the products from acetone, and fibers drawn from the melt, bis-pyroglutamic amides and esters appear to form hydrogen bonded supramolecular associations. In contrast to the polymeric-like properties, these compounds are readily soluble in water, alcohol, and chlorinated solvents, and display low intrinsic viscosity (0.13 dL/g for PDA-GLP in DMAC at 35° C.), all of which provides additional evidence for an associated structure (Structure 3).

[chemical structure]

### EXAMPLE 3

N-Methyl-N′-pyroglutamyl-1,3-propanediamine was prepared by the reaction illustrated 5 in Scheme 3.

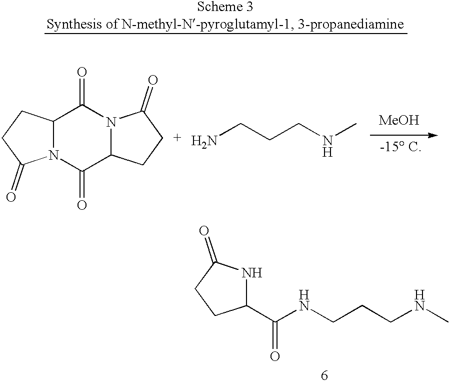

To a 100 mL round bottom flask were added pyroglutamic diketopiperazine (2.00 g, 0.0090 mol), MeOH (60 mL) and a magnetic stir-bar. The flask was cooled to −15° C. and N-methyl-1,3-diaminopropane (1.62 g, 0.0184 mol) was added and the reaction mixture was stirred for 12 hours while warming to room temperature. The solvent was evaporated in vacuo to give a clear oil. The oil slowly solidified after which the solid was broken up and dried at 60° C. under vacuum to give 6. Yield of 6 3.52 g (98%) white solid. ¹H NMR (DMSO-d₆ with TMS): δ: 8.02 (t, 1H), 7.83 (s, 1H), 3.98-3.91 (m, ¹H), 3.17-3.05 (m, 2H), 2.43 (t, 2H), 2.16-2.06 (m, 3H), 1.91-1.78 (m, 1H), 1.53 (m, 2H). ¹³C NMR(DMSO-d₆with TMS): δ: 177.4, 172.2, 55.9, 49.0, 36.9, 36.1, 29.3, 29.0, 25.4.

### EXAMPLE 4

N-Pyroglutamyl tetradecylamine was prepared by the reaction illustrated in Scheme 4.

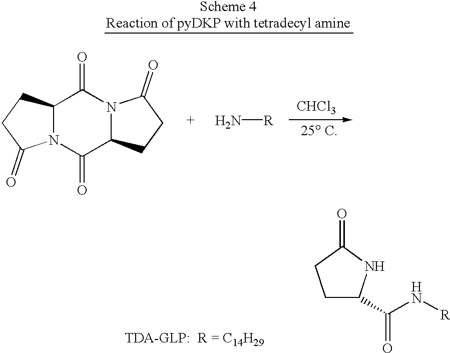

To a 250 mL round bottom flask were added pyroglutamic diketopiperazine (1.00 g, 0.0045 mol), CHCl₃ (100 mL), and a magnetic stir-bar. The flask was capped with a septa, purged with N₂ gas, and cooled to 0° C. in an ice bath. Tetradecylamine (1.97 g, 0.0092 mol) was added and the reaction was stirred for 12 hours while warming to room temperature. The solvent was removed in vacuo and the further dried under vacuum at 60° C. for 12 hours to give the product as a white solid. Yield 2.90 g (99.3%; mp=97-100° C.; ¹H NMR (CHCl₃-d/TFE): δ: 6.66 (s, 1H), 6.34 (s, ¹H), 4.08-4.02 (m, 1H), 3.16-3.09 (q, 2H), 2.47-2.25 (m, 3H), 2.06-1.97 (m, I1H), 1.40 (m, 2H), 1.17 (s, 24H), 0.78 (t, 3H). ¹³C NMR (CHCl₃-d/TFE): δ: 181.6, 172.9, 57.4, 40.1, 31.9, 29.7, 29.5, 29.4, 29.2, 29.1, 26.7, 25.6, 22.7, 13.8.

While the invention has been disclosed with respect to presently preferred embodiments, it will be appreciated that changes can be made without departing from the spirit of the invention. Accordingly, the scope of the invention is to be determined by the following claims rather than the foregoing description.

What is claimed is:

1. A process for forming amino acid derivatives from tricyclic diketopiperazines comprising the following reaction 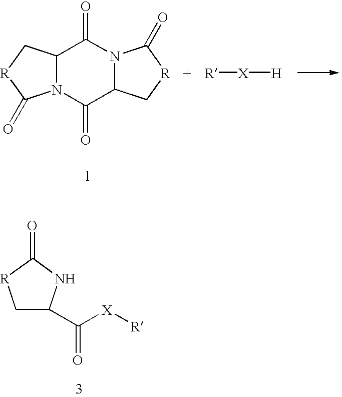
R=nothing, CH₂, 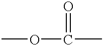
R′=alkyl, alkyl with heteroatoms and multiple functionality, aryl, aryl with heteroatoms and multiple functionality and combinations of these
X, Y=NH, O, S, and combinations of these

2. A process for forming bis-amino acid derivatives from tricyclic diketopiperazines comprising the following reaction 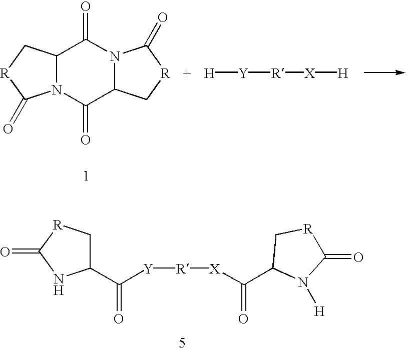
R=nothing, CH₂, 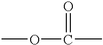
R′=alkyl, alkyl with heteroatoms and multiple functionality, aryl, aryl with heteroatoms and multiple functionality and combinations of these
X, Y =NH, O, S, and combinations of these

3. A process for forming reactive amino acid derivatives from tricyclic diketopiperazines comprising the following reaction 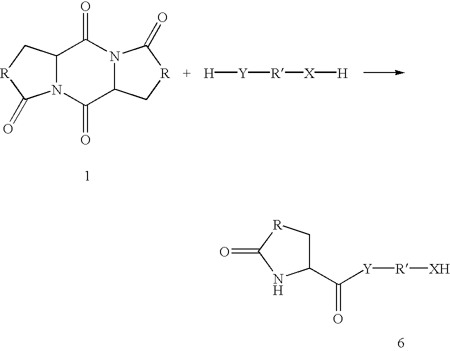
R=nothing, CH₂, 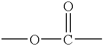
R′=alkyl, alkyl with heteroatoms and multiple functionality, aryl, aryl with heteroatoms and multiple functionality and combinations of these
X, Y=O, S, and combinations of these DOC)
